(19) United States
(12) Patent Application Publication
(10) Pub. No.: US 2006/0079924 A1
(43) Pub. Date: Apr. 13, 2006

(54) Apparatus for accessing a body cavity and methods of making same

(75) Inventors: Gerald J. Sanders, Sonoma, CA (US); Gerald Feuer, Atlanta, GA (US)

Correspondence Address: LUCE, FORWARD, HAMILTON & SCRIPPS LLP, 11988 EL CAMINO REAL, SUITE 200, SAN DIEGO, CA, 92130, US

(73) Assignee: FEMSPEC LLC, San Francisco, CA (US)

(21) Appl. No.: 11/245,506

(22) Filed: Oct. 7, 2005

Related U.S. Application Data
(63) Continuation-in-part of application No. 10/898,587, filed on Jul. 23, 2004.
Application No. 10/898,587 is a continuation-in-part of application No. 10/719,542, filed on Nov. 20, 2003.
(60) Provisional application No. 60/490,086, filed on Jul. 24, 2003.

(51) Int. Cl. A61M 29/00

(52) U.S. Cl. 606/192

(57) ABSTRACT

Apparatus is provided for accessing a body cavity comprising kit containing an annular member having a deflated insertion configuration and an inflated expanded configuration and an expandable support assembly including an upper lever coupled to a handle, wherein the expandable support assembly may be deployed within the inflatable body to enhance the radial strength of the inflatable body and provide an unobstructed view.

### REFERENCE TO RELATED APPLICATIONS

This application is a continuation-in-part of U.S. patent application Ser. No. 10/898,587, filed Jul. 23, 2004, which is incorporated herein by reference in its entirety.

### FIELD OF THE INVENTION

The present invention relates to methods and apparatus for accessing a body cavity, and more particularly, to methods and apparatus for gaining access to the female urogenitary tract.

### BACKGROUND OF THE INVENTION

Examination of the vagina and its associated anatomy is typically performed using a speculum, which provides access to the vagina by dilating the vaginal canal and then holding it in an expanded state. As currently used, a conventional speculum consists of a pair of metal jaws that are inserted into the vaginal canal and then actuated to expand the canal. For most patients, insertion and operation of the speculum is uncomfortable and may cause the patient to become tense, thus making a thorough examination difficult, if not impossible.

An example of an early speculum design is found in U.S. Pat. No. 196,600 to Shiland. The described apparatus is comprised of a number of rings secured “by solder or otherwise” between two bars. This device suffered from several disadvantages, not the least of which include patient discomfort from exposure to the bare metal, as well as potential injuries during use of the device, such as from interaction between tissue and the speculum's exposed articulating surfaces.

More recent articulating specula designs have employed resins in the product manufacture to overcome some patient discomfort due to exposure to bare metal. See, for example, U.S. Pat. Nos. 6,527,710 and 6,740,031 to Davidson, et al. Nevertheless, these designs may still expose a patient to some risk of discomfort from exposure to articulating surfaces.

Speculums having inflatable exterior walls have been developed, such as described in U.S. Pat. No. 5,716,329 to Dieter. The speculum described in that patent includes a rigid interior wall and an inflatable exterior wall that may be inflated with fluid after insertion to alleviate discomfort associated with expansion of the vaginal canal. The device described in that patent, however, is fairly complicated and because it combines both reusable and disposable components, may not be commercially practicable.

In view of the low cost needed to have a commercially viable disposable speculum product, others have attempted to develop speculums that comprise inflatable sacs or ribs, such as described in International Patent Publication No. WO97/24975 and Dutch Patent No. 9100599. The products described in these publications and patents do not appear to possess sufficient strength in the deployed position to be practical for widespread use, however.

U.S. Pat. No. 5,743,852 to Johnson describes an inflatable speculum comprising an inflatable cone-like structure comprising inner and outer wall elements that are sealed together along their edges, and which further includes a grid of contact areas comprising a grid pattern. That patent describes an insertion rod disposed within the speculum to assist in insertion, and is coupled to an external sheath that is withdrawn through the central lumen of the device when the insertion rod is withdrawn. A cone-shaped structure may be inserted within the inflated speculum once it is inflated to retain the speculum in the expanded state, and in addition, to provide support for a fiber-optic light or other instruments.

The foregoing Johnson patent appears to provide a number of advantages with respect to other inflatable speculum designs. However, the configuration of the insertion rod and sheath are expected to be problematic, in that the sheath is drawn from the distal (nearest the gynecologist) to the proximal edge (furthest within the patient) during removal, and may cause undue rubbing and discomfort. In addition, because the internal support structure disclosed in that patent does not extend to the proximal end of the speculum, it is possible for the forces applied by the patient's body to partially collapse the proximal end of the speculum. Finally, the use of sealed edges along the periphery of the inner and outer wall elements, especially at the proximal end of the speculum, may create a relative rigid structure capable of scraping the patient's cervix and causing patient discomfort.

U.S. Patent Publication US2003/0199737 to Deslauriers et al. describes an inflatable speculum having a plurality of longitudinally extending ribs arranged to delimit trapezoidal prisms within the volume of the speculum. As in the above-described WO publication and Dutch patent, the presence of the longitudinal ribs in the Deslauriers device is expected to preferentially distort to a central lumen of the speculum to a narrow ellipse when deployed, rather than providing a substantially circular lumen.

In view of the aforementioned drawbacks of previously known devices, it would be desirable to provide methods and apparatus for accessing a body cavity that is small, easy to insert into the body cavity and comfortable once inserted and actuated within the body cavity.

It further would be desirable to provide methods and apparatus for accessing a body cavity that provides sufficient strength to expand the body cavity while using low-cost materials that permit the apparatus to be discarded after a single use.

It also would be desirable to provide apparatus for accessing a body cavity that provides sufficient radial strength to expand a body cavity in the vast majority of cases, but which may include a further optional component for use in special situations, e.g., in examining or treating obese patients.

It still further would be desirable to provide methods for manufacturing apparatus to access a body cavity that substantially eliminate the presence of longitudinal ribs or features that cause preferential bending of the device in the inflated state, and thereby ensure a substantially circular working lumen.

It also would be desirable to provide methods for manufacturing apparatus to access a body cavity that substantially eliminate the presence of welds or seals along the proximal peripheral edges of the device, thereby reducing the risk of patient discomfort.

### SUMMARY OF THE INVENTION

In view of the foregoing, it is an object of the present invention to provide apparatus for accessing a body cavity that is small, easy to insert into the body cavity and comfortable once inserted and actuated within the body cavity.

It is another object of this invention to provide methods and apparatus for accessing a body cavity that provide sufficient strength to expand the body cavity while using low-cost materials that permit the apparatus to be discarded after a single use.

It is another object of this invention to provide apparatus for accessing a body cavity that provides sufficient radial strength to expand a body cavity in the vast majority of cases, but which may include a further optional component for use special situations, e.g., in examining or treating obese patients.

It is a further object of the present invention to provide methods for manufacturing apparatus to access a body cavity that substantially eliminate the presence of longitudinal ribs or features that cause preferential bending of the device in the inflated state, thereby ensuring a substantially circular working lumen.

It is yet another object of this invention to provide methods for manufacturing apparatus to access a body cavity that substantially eliminate the presence of welds or seals along the proximal peripheral edges of the device, thereby reducing the risk of patient discomfort.

In accordance with the principles of the present invention, apparatus is provided for accessing a body cavity comprising a kit containing an inflatable body and an optional expandable support member. The inflatable body is formed from a single sheet of material that is everted upon itself and sealed along its proximal edge (nearest the physician), thereby eliminating the presence of a distal seal or weld zone and providing an atraumatic proximal end. The inflatable body is inserted into the body cavity in a deflated configuration and then inflated to an expanded configuration, thereby expanding the walls of the body cavity.

The inflatable body includes a plurality of contact points arranged in a staggered pattern to permit substantially uniform pressure distribution within the inflatable body during expansion. In accordance with the principles of the present invention, the contact areas are arranged so as not to create substantially longitudinal features, but instead provide a substantially circular central lumen when the inflatable body is inflated under load.

The inflatable body is coupled to an inflation device, such as a bulb or pump, via a length of relatively stiff tubing that extends into and terminates within the inflatable body. The tubing is sufficiently rigid to permit the physician to exert a force of the inflatable body, in the contracted delivery configuration, to drive the inflatable body into the patient's orifice. In addition, a retractable, pre-lubricated sheath may be disposed on the exterior of the inflatable body to assist in inserting the device into the patient's orifice.

The optional expandable support assembly comprises at least two surfaces that are movable with respect to each other, but which remain shielded from patient contact by the inflatable body. In one embodiment, the expandable support assembly is capable of insertion and removal relative to the inflatable body, and may be inserted within the central lumen of the inflatable body after inflation of that component. The expandable support assembly then is deployed within the central lumen to enhance the rigidity of the inflatable body. Preferably, the expandable support assembly is formed of inexpensive plastic components and is manually operated by the physician. In a preferred embodiment, an upper lever is coupled to a handle by a plurality of hinged supports rod, and may be especially useful in conducting examination of obese patients.

Optionally, the apparatus may include an additional fixed internal support member as part of the kit in lieu of the expandable support assembly. The fixed support member preferably comprises an inexpensive plastic component that is mounted on a dilator, and is placed within the speculum to enhance the radial strength of the apparatus after deployment of the inflatable body. This fixed support member may be routinely employed with patients of average weight to seat the inflatable body after it is partially deployed.

In some embodiments, the inflatable body may include one or more pockets disposed within the central lumen of the inflatable body to permit a fiber-optic light or other instrument to be retained within the lumen. Alternatively, the inflatable body may include additional lengths of tubing that extend to a position near the proximal end of the apparatus to permit the evacuation of smoke generated during treatment of the organ, e.g., such as during leep-conization.

In still further alternative embodiments, the apparatus includes a handle assembly that may be attached to the inflatable body to facilitate insertion of the inflatable body into the body cavity, or to re-orient the field of view accessible through the central lumen of the inflatable body.

Methods of manufacturing the apparatus of the present invention also are provided.

### BRIEF DESCRIPTION OF THE DRAWINGS

The above and other objects and advantages of the present invention will be apparent upon consideration of the following detailed description, taken in conjunction with the accompanying drawings, in which like reference characters refer to like parts throughout, and in which:

FIG. 1 is a side view of the apparatus of the present invention in a deflated configuration;

FIGS. 2A and 2B are, respectively, cross-sectional views of the apparatus of FIG. 1 taken along view line 2-2 in the deflated and inflated states;

FIGS. 3A and 3B are, respectively, a side view and an end perspective view of the inflatable body of FIG. 1 in the inflated state;

FIG. 4 is a perspective view of a sheath for facilitating delivery of the inflatable body;

FIG. 5 is a perspective view of the expandable support assembly of FIG. 1;

FIGS. 6A-6C are, respectively, perspective views of the upper lever, handle and support rod of the expandable support assembly of FIG. 5;

FIGS. 7A-7B are side views depicting an alternative design of the inflatable body of FIG. 1 including an optional side pocket and aspiration tube;

FIG. 8 is a side view, partly in section, of a fixed support member and dilator for use with the apparatus of the present invention;

FIG. 9 is a perspective view of a preferred method of rolling the inflatable body to reduce patient discomfort during deployment;

FIG. 10 is a flow chart describing a preferred process for manufacturing the apparatus of FIG. 1;

FIG. 11 is a side view of the inflatable body of the present invention including a coating of a drug or other bioactive substance; and

FIG. 12 is a cross-sectional view of an alternative embodiment of the inflatable body of the present invention suitable for delivering bioactive substances.

### DETAILED DESCRIPTION OF THE INVENTION

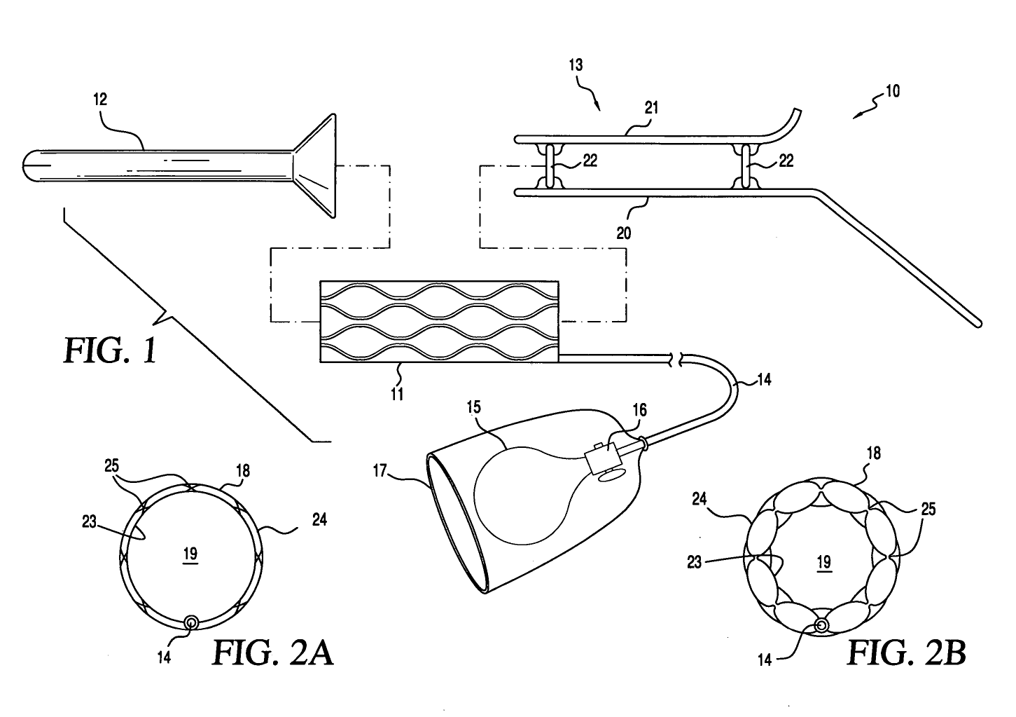

Referring to FIGS. 1-3, apparatus of the present invention is described that provides a low-cost single-use disposable apparatus kit for expanding a body cavity, such as the vaginal canal. Apparatus 10 comprises inflatable body 11, insertion sheath 12, and expandable support assembly 13. As described herein below, expandable support assembly 13 may be routinely used with patients in connection with inflatable body 12, or may be used only for patient's presenting special issues, such as obese patients for whom inflatable body 11 provides insufficient radial strength when deployed.

Inflatable body 11 includes inflation tube 14, inflator 15, valve 16 and shield 17. Inflatable body 11 transitions from a substantially flat tubular shape (FIG. 2A) to an expanded configuration (FIG. 2B) when inflated using inflator 15, illustratively a bulb. In the expanded configuration, inflatable body 11 forms annular main body portion 18 defining central lumen 19 that provides the physician with access to the interior of the body organ or lumen. In accordance with one aspect of the present invention, inflation tube 14 is bendable but is otherwise relatively stiff, so that force applied to the inflation tube may be used to push the inflatable body into a patient's orifice. Inflation tube 14 communicates with the interior of main body portion 18 to permit inflatable body 11 to be inflated and deflated.

Valve 16 preferably is a one-way valve that retains pressure within main body portion 18, but does not require that bulb 15 remain pressurized. Valve 16 may be selectively actuated to deflate main body portion 18. Bulb 15 and valve 16 preferably are coupled to inflation tube via a conventional luer fitting, so that these items may be uncoupled from inflatable body 11 and inflation tube 14 for subsequent reuse. Bulb 15 and valve 16 preferably are disposed within shield 16, e.g., a plastic bag, to prevent contamination with the patient's body fluids.

Insertion sheath 12 comprises a light-weight plastic sheath that restrains inflatable body 11 in a contracted position to facilitate insertion in the patient's organ or lumen. Sheath 12 includes a split bullet-nosed atraumatic shape that assists in insertion of the device, and is retracted distally over inflation tube 14 during deployment of the inflatable body.

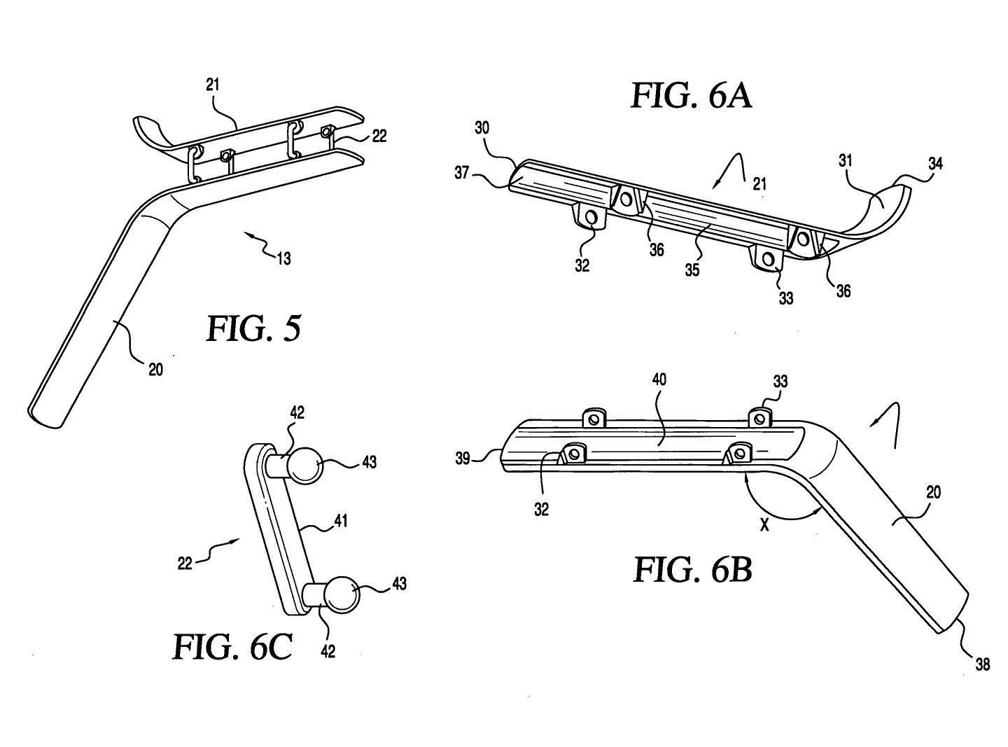

Expandable support assembly 13 comprises handle 20, upper lever 21, and a plurality of hinged support rods 22, as discussed further below with respect to FIGS. 5 and 6.

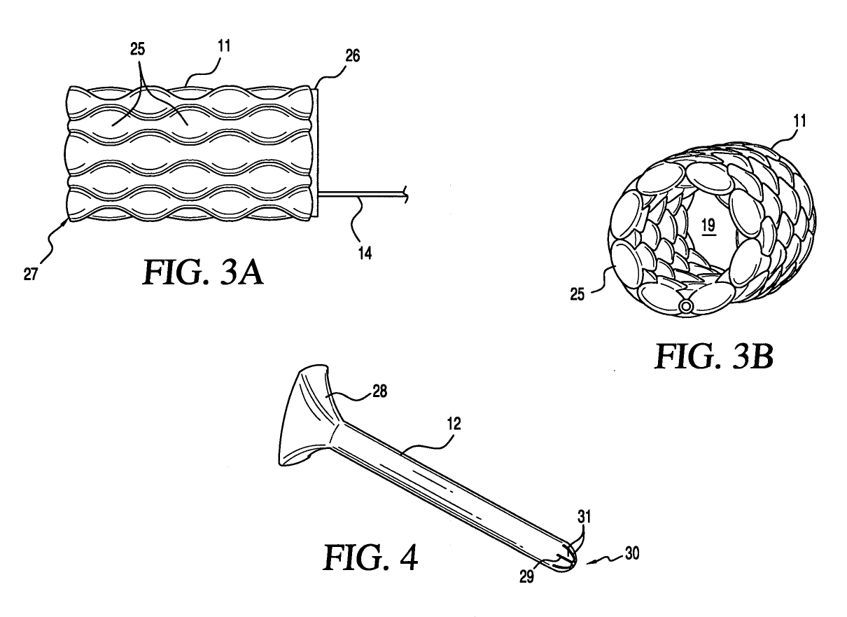

Referring to FIGS. 2 and 3, inflatable body 11 preferably comprises a polymeric, latex-free material and is formed so that exterior wall 23 is joined to interior wall 24 at plurality of pillow-like quilted contact areas 25. Preferably, contact areas 25 are arranged in a uniform pattern to allow for substantially uniform pressure distribution within the inflatable body 11 during expansion. In a preferred embodiment, 16 rows of contact areas are provided around the circumference of the inflatable body and axially staggered, i.e., longitudinally offset.

In accordance with the principles of the present invention, distributing the rows of contact areas 25 in an axially offset or staggered arrangement avoids the creation of longitudinal features on the inflatable body. Such features, which are present in the previously known devices, lead to preferential bending of the device under load, and permit the central lumen 19 to become distorted into a narrow ellipse. The offset grid pattern illustrated in FIG. 1, however, enhances the radial stiffness of the inflatable body in the expanded configuration, and ensures that central lumen 19 remains substantially circular in the inflated state, even under load.

Referring to FIG. 3, inflatable body 11 preferably comprises a single piece of material that is everted onto itself to form a double-layer tubular annulus that is approximately half as long as the original piece of material. In this manner, seam or weld 26 is formed only along one end of the inflatable body, as indicated in FIG. 3A, preferably at the proximal end of the inflatable body (nearest the physician). This avoids the presence of a seam or weld at distal end 27 of the inflatable body, and instead provides a soft, pillow-like atraumatic proximal end that reduces the risk of scraping or injuring tissue within the organ or lumen, as shown in FIG. 3B.

With respect to FIG. 4, insertion sheath 12 comprises a soft polymer tube, such as heat-shrinkable tubing, that retains inflatable body 11 in a contracted insertion configuration. Sheath 12 includes distal flange 25 to provide the physician with a grip to grasp and withdraw the sheath distally. Sheath 12 also includes slots 29 in bullet-shaped nose 30 that permits leaves 31 between slots 29 to open outward during retraction of the sheath.

Insertion sheath 12 preferably is lubricated with a biocompatible lubricant and then inserted into a patient's body cavity, e.g., the vagina. In accordance with one aspect of the present invention, inflation tube 14 is sufficiently rigid that it permits the physician to hold the inflatable body stationary within the body cavity with one hand, while retracting the insertion sheath from the inflatable body in a distal direction with the other hand. The insertion sheath is then removed over the luer at the distal end of inflation tube 14, and valve 16, bulb 15 and shield 17 then are coupled to the luer to permit the inflatable body to be inflated. Optionally, shield 17 may be omitted.

Referring now to FIGS. 5 and 6, expandable support assembly 13 in accordance with the present invention is described in greater detail. Applicants have observed during testing that in a certain segment of the population, especially obese women, the inflatable body 11 of the present invention may not provide sufficient radial strength to allow a clear field of view through the central lumen 19. Expandable support assembly 13 is configured to be deployed within inflatable body 11 after inflatable body 11 is inserted and partially deployed, to provide increased radial strength of inflatable body 11 and permit an unobstructed view through central lumen 19.

In a preferred embodiment, expandable support assembly 13 includes handle 20 coupled to upper lever 21 by hinged support rods 22. Handle 20 functions as a lower lever with which the physician can manipulate the deice to fully spread open inflatable body 11. Support rods 22 are coupled to handle 20 and upper lever 21 near the lateral edges of those structures to avoid obstructing central lumen 19 when deployed. Each of components 20-22 preferably is constructed of inexpensive molded plastic to reduce manufacturing costs. Other embodiments may reduce manufacturing costs further by adding throughwall apertures in handle 20 or upper lever 21 to reduce the material required for construction.

Upper lever 21 includes proximal end 30 (nearest from physician), distal end 31 (furthest from the physician), hinges 32 and 33 and grip 34. Support rods 22 engage within hinges 32 so that the support rods can pivot from a closed position aligned with surface 35 of upper lever 21 to an open position substantially orthogonal to upper lever 21 (as depicted in FIG. 5). Upper lever 21 is moved from the closed to the open position by applying a force to grip 34 that is directed away from handle 20. Because expandable support assembly 13 is placed within lumen 19 of inflatable body 11 after the inflatable body is partially expanded, it may be desirable to assist the initial separation of upper lever 21 from handle 20 to avoid the use of undue force on grip 34.

Distal hinges 32 may be taller than proximal hinges 33 to adjust the degree of separation between upper lever 21 and handle 20 in the closed position. Accordingly, when force is applied to grip 34 upper lever moves easily from the closed to the open position. Hinges 32 and 33 also include stop plates 36. If upper lever 21 was free to move relative to handle 22, it could be unstable at the point at which the two structures are furthest apart from each other. In a preferred embodiment, stop plates 36 prevent upper lever 21 from rotating more than a specified angle past the distance of maximum separation. Once support rods 22 contact stop plates 36, the compressive force applied by inflatable body 11 and body cavity act to retain expandable support assembly 13 in the open position.

Upper lever 21 also may include concave channel 37 located in at least a portion of the area medial to hinges 32 and 33, which provides the physician with a larger path through expandable support assembly 13 when deployed. In addition, grip 34 preferably includes an angled portion to improve manipulability. Grip 34 optionally may include variations in shape, texture, or material to further enhance manipulability.

With respect to FIG. 6B, handle portion 20 includes proximal end 38, distal end 39 and hinges 32 and 33 as described above with respect to upper lever 21. Handle 20 preferably also includes concave channel 40 located in at least a portion of the area medial to hinges 32 and 33 to enhance the physician's view through the expandable support assembly when deployed in inflatable body 11.

When deploying expandable support assembly 13, handle 20 is held in stationary while the physician manipulates upper lever 21 using grip 34. Preferably, handle 20 is longer than grip 34, thereby allowing the physician an increased surface to hold handle 20 while moving the upper lever. Likewise, it is preferred that the distal portion of handle 20 be positioned at angle X relative to the proximal section of the handle, where angle X is greater than 90 degrees more preferably between 120 and 160 degrees. Handle 20 optionally may also include variations in shape, texture, or material.

Referring now to FIG. 6C, support rod 22 preferably is constructed of inexpensive plastic, yet contains sufficient strength to not fracture or significantly deform when exposed to forces reasonably present during use. Support rod 22 illustratively comprises body 41, arms 42, and balls 43. Support rods 22 are attached at one end to handle 20 and at the other end to upper lever 21. Support rod 22 is coupled to handle 20 and upper lever 21 by passing balls 43 through opening in hinges 32 and 33. Balls 43 preferably are slightly larger than the opening in hinges 32 and 33, so that the support rods may be installed with moderate pressure, but are resistant to removal.

One method of using the components of the kit of the present invention of FIG. 1 is now described. First, inflatable body 11, disposed within insertion sheath 12 is inserted into the vagina. Inflation tube 14 is held stationary while the insertion tube is withdrawn distally. Bulb 15, valve 16 and shield 17 are attached to the luer termination of inflation tube 14 and the inflatable body 11 is partially inflated. Expandable support assembly 13 is placed in the closed position within lumen 19 of inflatable body 11. Force then is applied to upper lever 21 while holding handle 20 stationary to move upper lever 21 to the open position.

After upper lever 21 rotates beyond the point of maximum separation, support rods 22 contact stop plates 36. Inflatable body 11 then may be fully inflated to provide the physician with an unobstructed view of the cervix. Expandable support assembly 13 is held open by the compressive forces of inflatable body 11 and the patient's vagina. Physician can then continue to perform the desired examination or treatment. Alternatively, inflatable body 11 may be fully inflated prior to insertion and deployment of expandable support assembly 13.

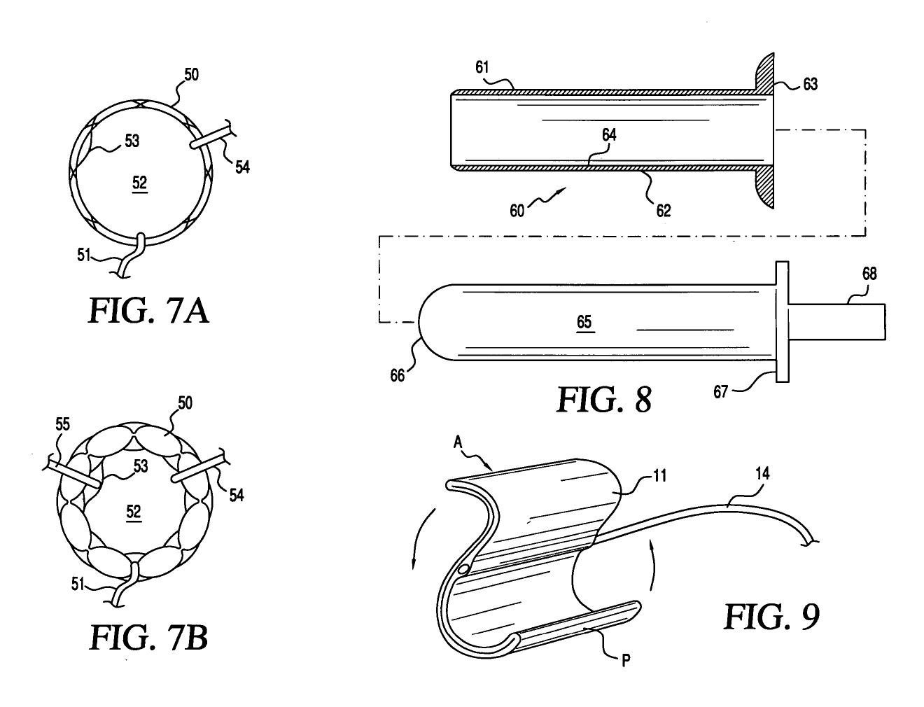

Referring now to FIGS. 7A and 7B, an alternative embodiment of the inflatable body of the present invention, suitable for use in colposcopy, leep-conization or other procedures, is described. Colposcopy is a procedure that looks at the cervix and vagina using glasses or other optical devices and generally requires vaginal illumination. Leep-conization is a procedure wherein an electrically-powered snare is used to remove tissue from an interior surface of the patient's cavity or organ, and can lead to the generation of smoke that must be evacuated to provide the physician with a clear field of view.

In FIGS. 7A and 7B, inflatable body 50 includes inflation tube 51, central lumen 52 having pocket or channel 53 and evacuation tube 54 fitted along its length. Channel 53 may be used to secure tool 55, such as fiber optic light source or other instrument, in position within central lumen 52. Evacuation tube 54 preferably extends the length of inflatable body 50 and includes a distal termination that permits tube 54 to be coupled to a suitable vacuum source to evacuate smoke or gases from within the body cavity during a procedure. Advantageously, channel 53 and evacuation tube 54 free up the physician's hands for other tasks. Alternatively, a light source to illuminate the body cavity may be substituted for evacuation tube 54 to facilitate procedures that require vaginal illumination, and channel 53 used to retain another instrument.

Referring to FIG. 8, optional fixed support member 60 of the present invention is described. in certain situations, it may be desirable to improve the field of view through the central lumen, but it may be impractical to use expandable support assembly 13 of FIGS. 1, 5 and 6. Fixed support member 61 therefore may be used in lieu of expandable support assembly 13, and comprises rigid disposable plastic tube 62 having proximal flange 63 and central lumen 64. Tube 62 is dimensioned to accept dilator 65 within central lumen 64. Dilator includes smooth distal end 66, flange 67 and handle 68. Flange 67 is configured to abut against flange 63, so that force exerted on handle 68 urges dilator 65 and tube 62 within the central lumen of the inflatable body of the apparatus of FIG. 1.

An illustrative use of the apparatus of FIGS. 1 and 8 as a vaginal speculum is now described. First, the inflatable body, disposed within insertion sheath 12 is inserted into the vagina. Inflation tube 14 then is held stationary while the insertion tube is withdrawn distally. Bulb 15, valve 16 and shield 17 then are attached to the luer termination of inflation tube 14 and the inflatable body is inflated. Dilator 65, with tube 62 disposed thereon, is then inserted into the central lumen of the inflatable body and driven forward by applying a proximally-directed force to handle 68. Once dilator 65 and tube 62 are fully inserted, dilator 65 is withdrawn, leaving tube 62 in position within the central lumen of the inflatable body. Advantageously, because tube 62 preferably extends to the proximal extremity of the inflatable body, it provides a clear field of view all the way to the patient's cervix.

With respect to FIG. 9, a preferred method of rolling the inflatable body of FIG. 1 to minimize discomfort during deployment is described. The present inventors have observed that in conventional jaw-type specula, the forces applied by the jaws are primarily in the anterior and posterior directions. This is believed to be so because lateral forces applied to the vagina are believed to cause discomfort. Accordingly, in accordance with one aspect of the present invention, the inflatable body is first flattened and then rolled in an S-shaped configuration having an anterior directed wing A and a posterior-directed wing P, as depicted in FIG. 11. When rolled in this manner, the forces applied to the vaginal walls during deployment of the inflatable body are primarily in the anterior and posterior directions, thereby reducing patient discomfort during inflation of the device.

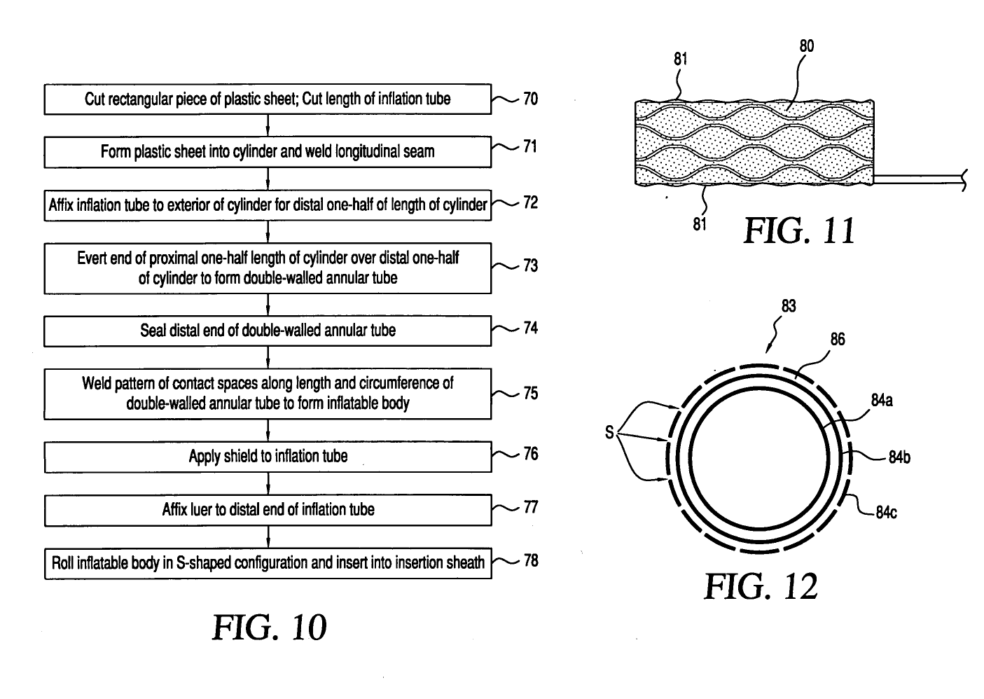

Referring now to FIG. 10, a method of making the apparatus of FIGS. 1-3 is described. At step 70, a rectangular piece of plastic sheet, such as 8 mil urethane, is cut to a desired size. For example, if the inflatable body is to be made having a nominal length of 12 cm and expanded diameter of 3.8 cm, the corresponding sheet size may be 15 cm×23 cm. At step 71, the sheet is formed into a cylinder, and a longitudinal seam is formed. At step 72, a length of inflation tube is affixed to exterior of cylinder for a distal one-half of length of cylinder. At step 73, the proximal one-half length of cylinder is everted over distal one-half of cylinder to form double-walled annular tube.

At step 74 a seal or weld is formed at the distal end of double-walled annular tube, thereby forming closed tube. As described hereinabove, having the weld only at the distal end of the inflatable body provides a smooth, atraumatic proximal end to the inflatable body. At step 75 a pattern of contact areas are formed along length and circumference of double-walled annular tube to form the inflatable body. As also described above, the contact areas are axially offset or staggered, so that when the inflatable body is inflated, no predominantly longitudinal features form that preferentially permit bending or partial collapse of the tube, as in previously known designs.

At step 76, shield 17 may be applied to the inflation tube, and at step 77, the luer termination may be applied to the distal end of the inflation tube. Alternatively, the luer termination may be applied, and the shield separately applied at a later time, e.g., after the insertion tube has been removed. Once the inflatable member is completed, at step 77, it may be rolled into an S-shaped configuration, as described above with respect to FIG. 9, and inserted into an insertion sheath at step 78. In subsequent steps, the insertion tube may be heated to cause it to shrink down on the inflatable body, and the device may then be packaged and sterilized.

With respect to FIG. 11, an alternative embodiment of the inflatable body of the present invention is described. Inflatable body 80 is similar in construction to the embodiment of FIG. 1 described above, but in addition includes coating 81 containing a drug, e.g., an antibiotic, for topical distribution within the body cavity or lumen. Alternatively, coating 81 may comprise a gene vector or protein coating. By providing the coating on the exterior wall, the drug, gene vector or protein may be delivered directly to the vaginal wall during examination and treatment. By way of example, coating 81 may contain Novocain, contraceptives, fertilization preparations, coagulants and various genes and proteins. Depending upon the pharmicokinetics of various drugs, genes and proteins and how they are absorbed in the vagina, coating 81 may contain more than one drug to be delivered into the vagina.

To facilitate delivery of the drug, gene or protein, features or patterns may be provided on the exterior wall. Alternatively, coating 81 may be lubricious and become slippery when exposed to water, thus reducing friction encountered during insertion of the device. As a further alternative, the apparatus may be pre-soaked in warm water prior to insertion to reduce patient discomfort, as the inflatable body is expected to retain some of the heat from the warm water.

In a further alternative embodiment depicted in FIG. 12, inflatable body 83 comprises interior layer 84a, middle layer 84b and exterior layer 84c. Interior layer 84a and middle layer 84b correspond to interior wall 23 and exterior wall 24 in the embodiment of FIG. 2A, while exterior layer 84c includes plurality of micro-perforations 85. The annulus between interior layer 84a and middle layer 84b is filled with gas or fluid to expand inflatable body 83, while the annulus between middle layer 84b and exterior layer 84c forms pocket 86, which may be filled with drugs, proteins or gene-vectors in a liquid or gel form. When the inflatable body is expanded inside a patient's body cavity, the drugs, proteins or gene-vectors within pocket 86 are forced through micro-perforations 85 and delivered to the wall of the body cavity.

With respect to the embodiments of FIGS. 11 and 12, coating 81 or pocket 86 may include medications for treating yeast infections, such as Terazol, Diflucan, Monistat and Gynazole. Alternatively, coating 81 or pocket 66 may include medications for treating bacterial infections, such as flagy and cleocin.

Although preferred illustrative embodiments of the present invention are described above, it will be evident to one skilled in the art that various changes and modifications may be made without departing from the invention. It is intended in the appended claims to cover all such changes and modifications that fall within the true spirit and scope of the invention.

What is claimed is:

1. Apparatus kit for accessing a body cavity, comprising:
an inflatable body consisting of a substantially cylindrical annular member, the annular member having a proximal end, a distal end, a central lumen, an interior, an inflation tube disposed in communication with the interior, and a plurality of staggered contact areas, the annular member formed by everting a length of material upon itself to define a main body portion having a circumferential seam only at proximal end, the annular member having a deflated configuration for insertion into the body cavity and an expanded configuration when inflated; and
an expandable support assembly comprising a handle and an upper lever coupled to the handle by a plurality of support rods, the expandable support assembly having a closed position and an open position,
wherein the expandable support assembly is configured to be inserted within the central lumen of the annular member to support the inflatable body in the expanded configuration.

2. The apparatus of claim 1, wherein the distal end of the annular member is configured to protect a patient's cervical area from rubbing or scraping.

3. The apparatus of claim 1, wherein the everted material forms an interior wall and an exterior wall and the inflation tube is interposed between the interior wall and the exterior wall.

4. The apparatus of claim 1, wherein the inflation tube is sufficiently rigid to transmit force when the annular member is in the deflated configuration.

5. The apparatus of claim 1, further comprising a channel disposed within the central lumen, wherein the channel facilitates securing tools during a procedure.

6. The apparatus of claim 1, further comprising an inflator coupled to the inflation tube.

7. The apparatus of claim 6, further comprising a one-way valve interposed between the inflator and the annular member.

8. The apparatus of claim 1, wherein the annular member further comprises a lubricous exterior coating.

9. The apparatus of claim 1 further comprising a shield to reduce contamination of the inflator.

10. The apparatus of claim 1 wherein the plurality of contact areas do not form longitudinal features when the inflatable body is in the expanded configuration.

11. The apparatus of claim 1, wherein the annular member further comprises a coating for topical application within the body cavity.

12. The apparatus of claim 11, wherein the coating contains drugs, gene vectors or proteins.

13. The apparatus of claim 11, wherein the coating includes a medication for treating yeast infections.

14. The apparatus of claim 1, wherein the annular member further comprises an exterior layer including a plurality of micro-perforations for the passage of drugs in a liquid or gel form.

15. The apparatus of claim 1, further comprising a sheath that surrounds the inflatable body during insertion into the body cavity.

16. The apparatus of claim 1, further comprising a fixed support member that may be inserted within the annular member to enhance the rigidity of the annular member.

17. The apparatus of claim 1 wherein plurality of support rods are coupled to the upper lever and the handle by hinges.

18. The apparatus of claim 17 wherein at least some of the hinges are taller than others to improve ease of operation of the expandable support assembly.

19. The apparatus of claim 18 wherein the hinges include stop plates that limit a range of motion of the support rods.

20. The apparatus of claim 1 wherein at least one of the upper lever or handle includes a concave channel to improve visibility through the expandable support assembly in the open position.
